(19) United States
(12) Patent Application Publication
(10) Pub. No.: US 2022/0322785 A1
(43) Pub. Date: Oct. 13, 2022

(54) SHOE INSOLE AND SHOE WITH INSOLE STRUCTURE

(71) Applicant: Itaru Nakazawa, Nankoku-shi, Kochi (JP)

(72) Inventor: Itaru Nakazawa, Nankoku-shi, Kochi (JP)

(73) Assignee: Itaru Nakazawa, Nankoku-shi, Kochi (JP)

(21) Appl. No.: 17/634,377

(22) PCT Filed: Mar. 10, 2021
(86) PCT No.: PCT/JP2021/009637

§ 371 Date: Feb. 10, 2022

(30) Foreign Application Priority Data
Jun. 5, 2020 (JP) 2020-098771

(51) Int. Cl. A43B 7/14; A43B 17/00

(52) U.S. Cl. CPC A43B 7/14; A43B 17/00

(57) ABSTRACT

A shoe insole and a shoe with an insole structure, which are capable of, concerning the foot arch structure, preventing fallen arches, restoring and correcting the arches, as well as allowing the wearers to maintain the sense of balance. The shoe insole comprises an insole main body having a substantially foot shape; a lower cushioning portion provided on the insole main body in a front portion thereof for supporting a part from the tiptoe to the position of the metatarsal head part; an upper cushioning portion having a substantially U shape provided on the lower cushioning portion for supporting a part corresponding to the positions of the toe, the first toe ball, and the fifth toe ball; and a heel supporting portion provided on the insole main body in a back portion thereof for supporting a portion corresponding to the position of the heel.

### TECHNICAL FIELD

The present invention relates to a shoe insole and a shoe with an insole structure, and in particular to a shoe insole and a shoe with an insole structure suitable for, concerning the foot arch structure, the prevention of fallen arches, recovery and correction of the arches, and also suitable for posture correction.

### BACKGROUND ART

The human foot has three arch structures: the inner longitudinal arch, the outer longitudinal arch, and the transverse arch. These three arch structures have a very important function in walking. They play, for example, the role of cushioning to absorb shocks in walking, the role of a spring when taking a next step, and the role of maintaining stable standing and stable walking postures. However, when the arches lower, these functions are not ensured, thus resulting in gait disturbance, arthropathy, pain in the sole of the foot, as well as other various foot disorders such as hallux valgus, flat feet, or metatarsus latus (broad foot). Therefore, it is very important to prevent fallen arches, and restore and correct the arches.

Accordingly, various shoe insoles with a structure for supporting the foot arch structure have been disclosed (see, for example, Patent Literature 1 to Patent Literature 3). Patent Literature 1 discloses a shoe insole with an arch portion, which is characterized by having a contour of the entire sole and including an elastic or viscoelastic pad made up of, as an integral configuration, at least one of a gently-downward convex transverse arch pad portion, an inner longitudinal arch pad portion, and an outer longitudinal arch pad portion, that correspond to the transverse arch of the sole, the inner longitudinal arch of the sole, and the outer longitudinal arch of the sole, respectively.

Patent Literature 2 discloses an insole made of a plate-shaped flexible material as a whole, characterized in that an insole body having a flat shape corresponding to the sole of the foot includes, on its upper surface, a metatarsal raised portion integrally having (1) a central ridge portion raised to abut on the metatarsal arch portion of the second to the fourth toes, (2) an outer longitudinal arch ridge portion continuously that extends from the central ridge portion toward an outer longitudinal arch direction and that is raised to intersect a lower portion of the outer longitudinal arch, and (3) a first to second intermetatarsal raised portion that continuously extends from an inner vertex of the central ridge portion to the vicinity of the heads of the first and second metatarsals along the middle of the first and second metatarsals.

Further, Patent Literature 3 discloses a shoe insole including a base portion having a predetermined height and thickness and a constant step at a predetermined portion, and a predetermined projection portion on the base portion. The shoe insole is characterized in that the base portion includes, as an outer shape, a heel surface portion corresponding to the entire heel bottom surface, a narrow surface portion with a largely-hollowed plantar arch portion, and a top surface portion corresponding to a proximal phalanx, has an inner edge level difference from the inner side of the narrow surface portion to the rear edge of the top surface portion and a vertex level difference at the front edge of the top surface portion, and has the protrusion portions at the tail end and the inner and outer sides of the heel portion in the periphery of the base portion, and at the inner and outer sides of the vertex portion.

### CITATION LIST

### Patent Literature

Patent Literature 1: Japanese Laid-Open Patent Application Publication No. H11-226042
Patent Literature 2: Japanese Laid-Open Patent Application Publication No. 2010-275
Patent Literature 3: Registered Utility Model No. 3224343

### SUMMARY OF INVENTION

### Technical Problem

The shoe insole disclosed in Patent Literature 1 supports the arch of the foot by means of a viscoelastic part, the shoe insole disclosed in Patent Literature 2 supports the arch of the foot by means of a ridge part, and the shoe insole disclosed in Patent Literature 3 supports the arch of the foot by means of a narrow surface portion, a top surface portion, an inner edge level difference, a peripheral wall, and the like. In other words, previously-known insoles directly support the arches to stabilize the walking posture, alleviate gait disturbance, restore the arches, and prevent the fallen arches.

However, in the structure of Patent Literature 1 in which the arches are directly supported by a viscoelastic portion or the like, the movement of the arch is restricted, thereby hindering the arch structure to fully exert its original function, i.e., the cushioning function and the spring function mentioned above during walking.

In addition, although the direct support of the arch is expected to alleviate gait disturbance and prevent the fallen arches to some extent, it is considered difficult to obtain the effect of correcting one's own standing posture or walking posture by relying on the sensation of the soles.

Under such circumstances, the inventor of the present application conducted intensive study in light of the problems described above in order to provide a shoe insole and a shoe with an insole structure, which are capable of, concerning the foot arch structure, preventing fallen arches, restoring and correcting the arches, as well as allowing the wearers to maintain the sense of balance by relying on the sensation of the sole of the foot or enhance the sense of balance to correct the standing and walking postures by themselves. As a result, the inventor accomplished the present invention.

### Solution to Problem

More specifically, the invention of claim 1 is an shoe insole characterized by including an insole main body having a substantially foot shape; a lower cushioning portion provided on the insole main body in a front portion thereof for supporting a part from the tiptoe to the position of the metatarsal head part; an upper cushioning portion having a substantially U shape provided on the lower cushioning portion for supporting a part corresponding to the positions of the toe, the first toe ball, and the fifth toe ball; and a heel supporting portion provided on the insole main body in a back portion thereof for supporting a part corresponding to the position of the heel.

The invention of claim 2 is the shoe insole according to claim 1, wherein the height of a lamination of the lower cushioning portion and the upper cushioning portion is substantially identical to the height of the upper face of the heel supporting portion.

The invention of claim 3 is the shoe insole according to claim 1 or 2, which includes a toe supporting portion provided on the upper cushioning portion for supporting the toe.

The invention of claim 4 is an shoe insole characterized by including an insole main body having a substantially foot shape; a lower cushioning portion provided on the insole main body in a front portion thereof for supporting a part from the tiptoe to the position of the metatarsal head part; and a heel supporting portion provided on the insole main body in a back portion thereof for supporting a part corresponding to the position of the heel, wherein the insole main body includes a first toe hole formed in a part corresponding to a part from the IP joint to the position of the first toe ball, and a MP joint hole formed in a part corresponding to the positions of at least the second toe ball, the third toe ball, and the fourth toe ball, and a fifth toe ball hole formed in a part corresponding to the position of the fifth toe ball, and a heel hole formed in a part corresponding to the position of the calcaneus center.

The invention of claim 5 is the shoe insole according to claim 4, wherein the height of the upper face of the lower cushioning portion is substantially identical to the height of the upper face of the heel supporting portion.

The invention of claim 6 is the shoe insole according to claim 4 or 5, which includes a toe supporting portion provided on the lower cushioning portion for supporting the toe.

The invention of claim 7 is a shoe with an insole structure characterized by including a lower cushioning portion provided in a front portion of an upper face of a middle sole for supporting a part from the tiptoe to the position of the metatarsal head part; an upper cushioning portion having a substantially U shape provided on the lower cushioning portion for supporting a part corresponding to the positions of the toe, the first toe ball, and the fifth toe ball; and a heel supporting portion provided in a back portion of the upper face of the middle sole for supporting a part corresponding to the position of the heel.

The invention of claim 8 is the shoe with an insole structure according to claim 7, wherein the height of the lamination of the lower cushioning portion and the upper cushioning portion is substantially identical to the height of the upper face of the heel supporting portion.

The invention of claim 9 is the shoe with an insole structure according to claim 7 or 8, which includes a toe supporting portion provided on the upper cushioning portion for supporting a part corresponding to the position of the toe.

The invention of claim 10 is a shoe with an insole structure characterized by including a lower cushioning portion provided in a front portion of the upper face of the middle sole for supporting a part from the tiptoe to the position of the metatarsal head part; and a heel supporting portion provided in a back portion of the upper face of the middle sole for supporting a part corresponding to the position of the heel, wherein the middle sole includes a first toe hole formed in a part from the IP joint to the position of the first toe ball, and a MP joint hole formed in a part corresponding to the positions of at least the second toe ball, the third toe ball, and the fourth toe ball, and a fifth toe ball hole formed in a part corresponding to the position of the fifth toe ball, and a heel hole formed in a part corresponding to the position of the calcaneus center.

The invention of claim 11 is the shoe with an insole structure according to claim 10, wherein the height of the upper face of the lower cushioning portion is substantially identical to the height of the upper face of the heel supporting portion.

The invention of claim 12 is the shoe with an insole structure according to claim 10 or 11, which includes a toe supporting portion provided on the lower cushioning portion for supporting a part corresponding to the position of the toe.

### Advantageous Effects of Invention

The shoe insole and the shoe with an insole structure of the present invention enable, concerning the foot arch structure, prevention of fallen arches, and recovery and correction of the arches. In particular, instead of providing a structure for directly supporting arches, the present invention has the lower cushioning portion and the upper cushioning portion having a flat shape, thereby providing an effect of allowing the wearers to maintain a sense of balance by relying on the sensation in the sole of the foot, or to enhance the sense of balance and correct the standing and walking postures by themselves.

In addition, by adding a level difference between the lower cushioning portion and the upper cushioning portion in the shoe insole and the shoe with an insole structure of the present invention, it is possible to naturally support the portions of the second toe ball, the third toe ball, and the fourth toe ball that protrude to the backside of the foot to a greater extent than the first toe ball and the fifth toe ball during walking or standing on tiptoe, thereby preventing pain in the sole of the foot even during prolonged walking or during standing, thus suppressing gait disturbance and arthropathy.

Furthermore, with the shoe insole and the shoe with an insole structure of the present invention in which the height of the lamination of the lower cushioning portion and the upper cushioning portion are substantially identical to the height of the upper face of the heel supporting portion, it becomes possible to further enhance an effect of preventing fallen arches, maintaining a sense of balance, and correcting the postures.

Furthermore, with the shoe insole and the shoe with an insole structure of the present invention in which a toe supporting portion for supporting the part corresponding to the position of the toe is provided on the upper cushioning portion, it becomes easier to obtain the sense of kicking the ground with the toes during walking or the sense of grasping the ground with the toes in a standing posture, thereby improving stability of the posture and reducing fatigue in the sole.

In addition, by providing through holes (the first toe hole, MP joint hole, the fifth toe ball hole, the heel hole) at predetermined positions in the insole main body or in the middle sole of the shoe insole and the shoe with an insole structure of the present invention, the portions of the second toe ball, the third toe ball, and the fourth toe ball that protrude to the backside of the foot to a greater extent than the first toe ball and the fifth toe ball during walking or standing on tiptoe can slightly sink into the sole. As a result, the front portion of the sole can be naturally supported, thereby preventing pain in the sole of the foot even during prolonged walking, thus suppressing gait disturbance and arthropathy. In addition, even in a standing posture, the ball of each toe and the heel part slightly sunk into the through holes formed in the portions corresponding to the positions of the ball of each toe and the heel part; therefore, the anterior portion of the sole and the heel portion can be supported in a flat state, thereby alleviating or preventing pain in the sole.

During exercise, particularly upon the start or the end of exercise, a big load is applied to the first toe. However, by laminating the lower cushioning portion and the upper cushioning portion or by forming a first toe hole in the insole main body or in the middle sole in the shoe insole and the shoe with an insole structure of the present invention, the load applied to the first toe is greatly reduced, thereby alleviating or preventing pain in the sole.

Furthermore, compared with the embodiment of the present invention in which the lower cushioning portion and the upper cushioning portion are laminated, the thickness of the insole and the insole structure can be reduced in the embodiment in which through holes are formed in the insole main body or at predetermined potions of the middle sole according to the present invention.

Furthermore, in the embodiment in which through holes are formed in the insole main body or at predetermined potions of the middle sole in the shoe insole and the shoe with an insole structure of the present invention, by setting the height of the upper face of the lower cushioning portion to be substantially identical to the height of the upper face of the heel supporting portion, it becomes possible to further enhance an effect of preventing fallen arches, maintaining a sense of balance, and correcting the postures.

Furthermore, in the embodiment in which through holes are formed in the insole main body or at predetermined potions of the middle sole in the shoe insole and the shoe with an insole structure of the present invention, by providing a toe supporting portion for supporting the part corresponding to the position of the toe on the lower cushioning portion, it becomes easier to obtain the sense of kicking the ground with the toes during walking or the sense of grasping the ground with the toes in a standing posture, thereby improving stability of the posture and reducing fatigue in the sole.

### BRIEF DESCRIPTION OF DRAWINGS

FIG. 1 is a plan view of a shoe insole according to an embodiment of the present invention.

FIG. 2A is a right side view of the shoe insole shown in FIG. 1.

FIG. 2B is a cross-sectional view, taken along line A-A in FIG. 1.

FIG. 3A is a plan view showing a lower cushioning portion of the shoe insole shown in FIG. 1.

FIG. 3B is a plan view showing an upper cushioning portion of the shoe insole shown in FIG. 1.

FIG. 4 is a plan view showing a state in which a foot is placed on the shoe insole shown in FIG. 1.

FIG. 5 is a plan view of a shoe insole according to another embodiment of the present invention.

FIG. 6A is a right side view of the shoe insole shown in FIG. 5.

FIG. 6B is a cross-sectional view, taken along line B-B in FIG. 5.

FIG. 7 is a plan view showing a state in which a foot is placed on the shoe insole shown in FIG. 5.

FIG. 8 is a plan view of a shoe insole according to another embodiment of the present invention.

FIG. 9 is a plan view showing a positional relationship between an insole main body and a foot according to the shoe insole shown in FIG. 8.

FIG. 10 is a plan view showing a positional relationship between the insole main body, the lower cushioning portion, and the foot of the shoe shown in FIG. 8.

FIG. 11 is a plan view of a shoe insole according to another embodiment of the present invention.

### DESCRIPTION OF EMBODIMENTS

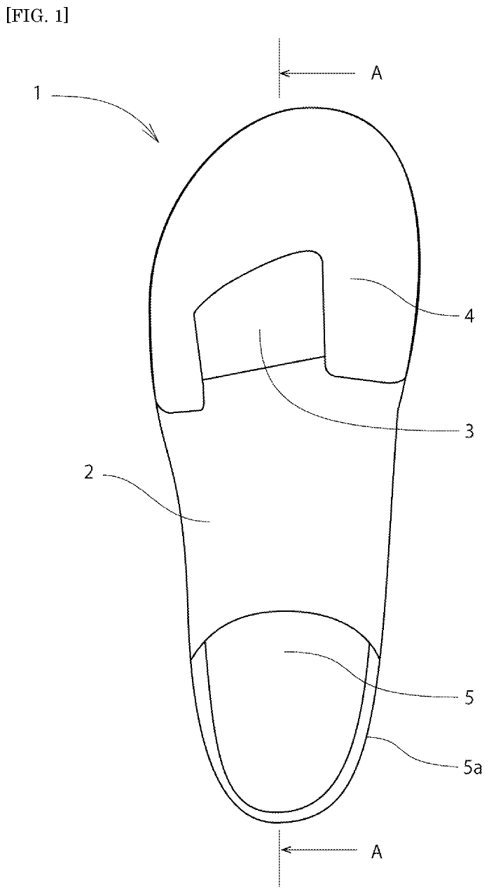

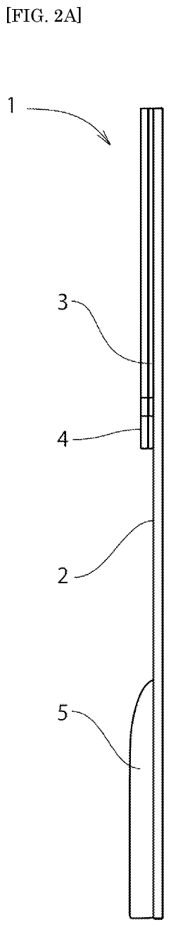

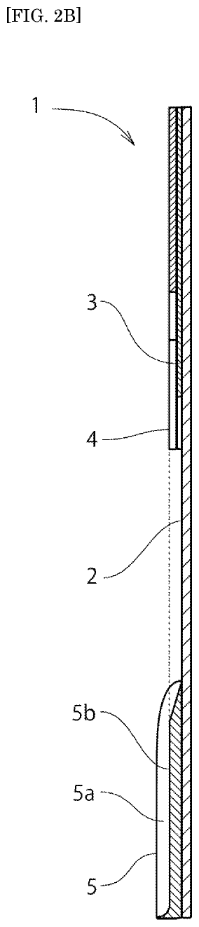

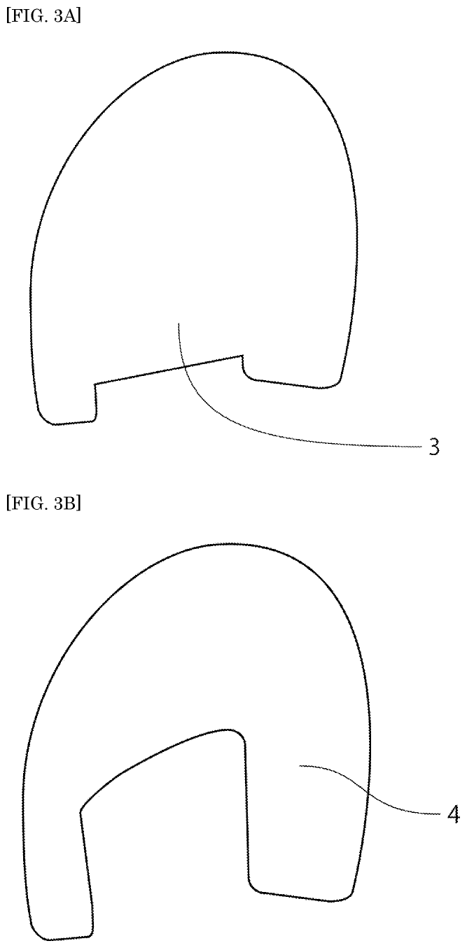

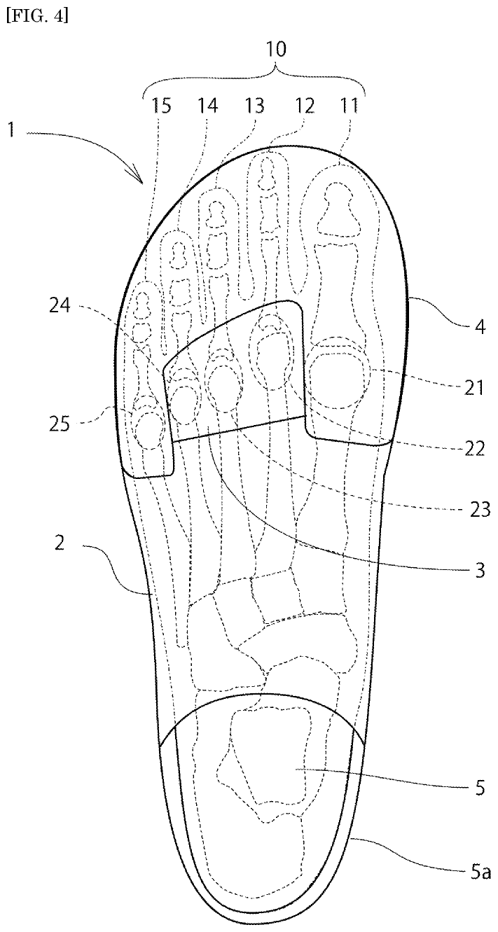

The shoe insoles and the shoes with an insole structure of the present invention are described below. First, an embodiment of a shoe insole in which the lower cushioning portion and the upper cushioning portion are laminated (hereinafter referred to as “a lamination type”) is described in detail based on the drawings. FIG. 1 is a plan view of a shoe insole 1 (for a left foot) according to one embodiment of the present invention. FIG. 2A is a right side view of the shoe insole 1 according to the present embodiment shown in FIG. 1. FIG. 2B is a cross-sectional view, taken along line A-A in FIG. 1. FIG. 3A is a plan view showing a lower cushioning portion 3 of the shoe insole 1 according to the present embodiment. FIG. 3B is a plan view showing an upper cushioning portion 4 of the shoe insole 1. FIG. 4 is a plan view showing a state in which a foot is placed on the shoe insole 1 according to the present embodiment.

As shown in FIGS. 1 to 4, the lamination type shoe insole 1 according to one embodiment of the present invention is characterized by including an insole main body 2 having a substantially foot shape; a lower cushioning portion 3 provided on the insole main body 2 in a front portion thereof for supporting a part from the tiptoe to the position of the metatarsal head part; an upper cushioning portion 4 having a substantially U shape provided on the lower cushioning portion 3 for supporting a part corresponding to the positions of the toe 10, the first toe ball 21, and the fifth toe ball 25; and a heel supporting portion 5 provided on the insole main body 2 in a back portion thereof for supporting a portion corresponding to the position of the heel.

The lower cushioning portion 3, which is provided on the insole main body 2 in a front portion thereof in the shoe insole 1 according to the present embodiment, has a flat shape as shown in FIG. 3A. The lower cushioning portion 3 planarly supports a part from the tiptoe to the position of the metatarsal head part, namely the toe 10, the first toe ball 21, the second toe ball 22, the third toe ball 23, the fourth toe ball 24 and the fifth toe ball 25.

As shown in FIG. 4, the first toe ball 21 in the sole of the foot is a bulging portion located at the first metatarsal head part that extends to the first toe 11, and the fifth toe ball 25 is a bulging portion located at the fifth metatarsal head part that extends to the fifth toe 15. Similarly, in the sole, the second metatarsal head part leading to the second toe 12 is referred to as a second toe ball 22, the third metatarsal head part that extends to the third toe 13 is referred to as a third toe ball 23, and the fourth metatarsal head part that extends to the fourth toe 14 is referred to as a fourth toe ball 24. Although the second toe ball 22, the third toe ball 23, and the fourth toe ball 24 do not have an apparent bulging portion unlike the first toe ball 21 or the fifth toe ball 25, the second metatarsal head part is referred to as a second toe ball 22, the third metatarsal head part is referred to as a third toe ball 23, and the fourth metatarsal head part is referred to as a fourth toe ball 24 in the present application for ease of explanation.

The upper cushioning portion 4 provided on the lower cushioning portion 3 according to the present embodiment has a substantially U shape in a plan view as shown in FIG. 3B, and supports the portions corresponding to the positions of the toe 10, the first toe ball 21, and the fifth toe ball 25. By providing the upper cushioning portion 4 having a substantially U shape on the lower cushioning portion 3, as shown in FIGS. 2 and 4, a lamination of the lower cushioning portion 3 and the upper cushioning portion 4 is formed in the portion where the toe 10, the first toe ball 21, and the fifth toe ball 25 are located. Further, the lower cushioning portion 3 is exposed in the portion where the second toe ball 22, the third toe ball 23, and the fourth toe ball 24 are located, thereby forming a level difference between the lower cushioning portion 3 and the upper cushioning portion 4.

Further, the heel supporting portion 5 provided in a back portion of the insole main body 2 according to the present embodiment supports the part corresponding to the position of the heel. Similar to the lower cushioning portion 3 or the upper cushioning portion 4, the heel supporting portion 5 may be formed in a flat shape; however, the heel supporting portion 5 preferably has a thick portion 5a that wraps around the heel to provide a stable support for the heel.

In contrast to the previously-known structure for directly supporting arches, the lamination type shoe insole 1 of the present embodiment with the structure described above has the lower cushioning portion 3 and the upper cushioning portion 4 having a flat shape. Therefore, the shoe insole 1 ensures an effect of allowing the wearers to maintain a sense of balance by relying on the sensation in the sole of the foot, or to enhance the sense of balance and correct the standing and walking postures by themselves, thereby preventing fallen arches or restoring and correcting the arches.

Further, in the shoe insole 1 of the present embodiment, by providing the upper cushioning portion 4 having a substantially U shape on the lower cushioning portion 3, the portion where the toe 10, the first toe ball 21, and the fifth toe ball 25 are located has a lamination of the lower cushioning portion 3 and the upper cushioning portion 4; accordingly, the toe 10, the first toe ball 21, and the fifth toe ball 25 are planarly supported directly by the upper face of the upper cushioning portion 4, and the lamination of the lower cushioning portion 3 and the upper cushioning portion 4 absorbs shocks from the ground, thereby reducing shocks applied to the toe 10, the first toe ball 21, and the fifth toe ball 25 during walking.

In addition, since the lower cushioning portion 3 is exposed in the portion where the second toe ball 22, the third toe ball 23, and the fourth toe ball 24 are located, thereby forming a level difference between the lower cushioning portion 3 and the upper cushioning portion 4, it is possible to naturally support the second toe ball 22, the third toe ball 23, and the fourth toe ball 24 that protrude to the backside of the foot to a greater extent than the first toe ball 21 and the fifth toe ball 25 during walking or standing on tiptoe, thereby preventing pain in the sole of the foot even during prolonged walking or during standing, thus suppressing gait disturbance and arthropathy.

Further, during exercise, particularly upon the start or the end of exercise, a big load is applied to the first toe. However, in the shoe insole 1 of the present embodiment, by laminating the lower cushioning portion 3 and the upper cushioning portion 4, the load applied to the first toe 11 and the first toe ball 21 is greatly reduced, thereby alleviating or preventing pain in the sole.

In this embodiment of the shoe insole 1, the heights (thicknesses) of the lower cushioning portion 3, the upper cushioning portion 4, and the heel supporting portion 5 are not particularly limited. However, as shown in FIG. 2B, the height (thickness) of the lamination of the lower cushioning portion 3 and the upper cushioning portion 4 is preferably substantially the same as the height of the upper face 5b of the heel supporting portion 5 (the thickness of the heel supporting portion 5). By thus setting the height of the lamination of the lower cushioning portion 3 and the upper cushioning portion 4 to a height substantially identical to the height of the upper face 5b of the heel supporting portion 5, it becomes possible to further enhance an effect of preventing fallen arches, maintaining a sense of balance, and correcting the postures.

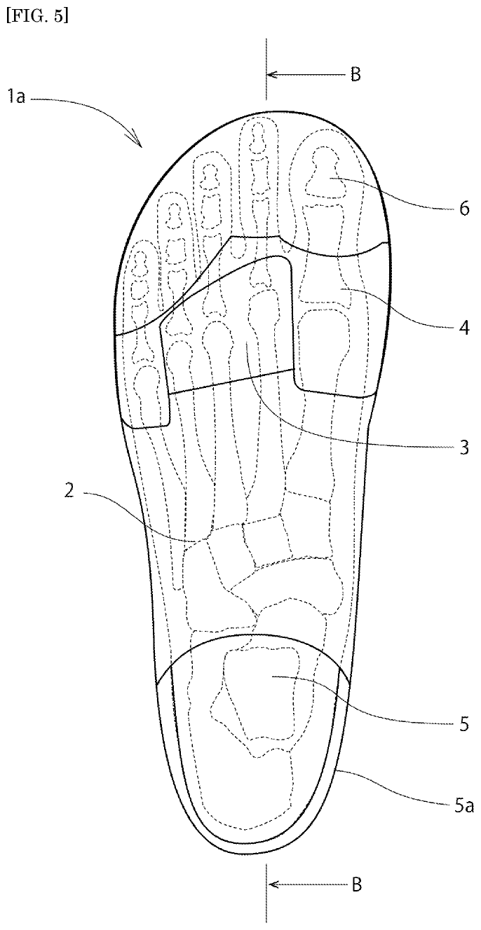

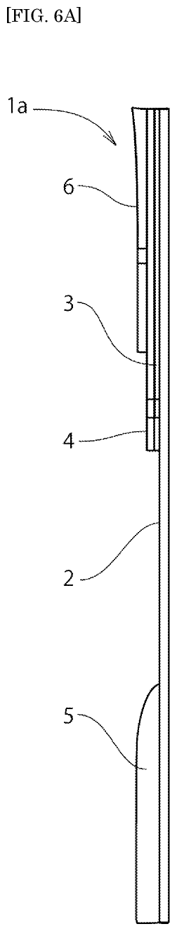

FIGS. 5 to 7 show a shoe insole la as another embodiment of the present invention. The shoe insole 1a is similar to the lamination type shoe insole 1 according to the embodiment described above in terms of its fundamental structure, and includes an insole main body 2 having a substantially foot shape; a lower cushioning portion 3 provided on the insole main body 2 in a front portion thereof for supporting a part from the tiptoe to the position of the metatarsal head part; an upper cushioning portion 4 having a substantially U shape provided on the lower cushioning portion 3 for supporting a part corresponding to the positions of the toe 10, the first toe ball 21, and the fifth toe ball 25; and a heel supporting portion 5 provided on the insole main body 2 in a back portion thereof for supporting a part corresponding to the position of the heel. Furthermore, in this embodiment of the shoe insole 1a, a toe supporting portion 6 for supporting the toe 10 is provided on the upper cushioning portion 4.

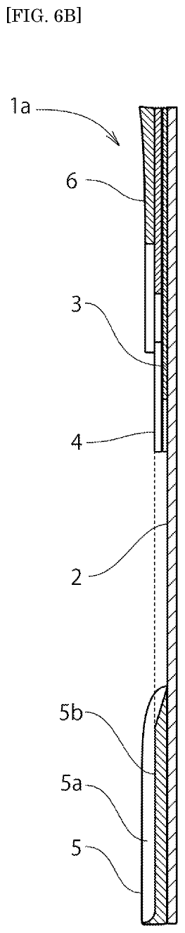

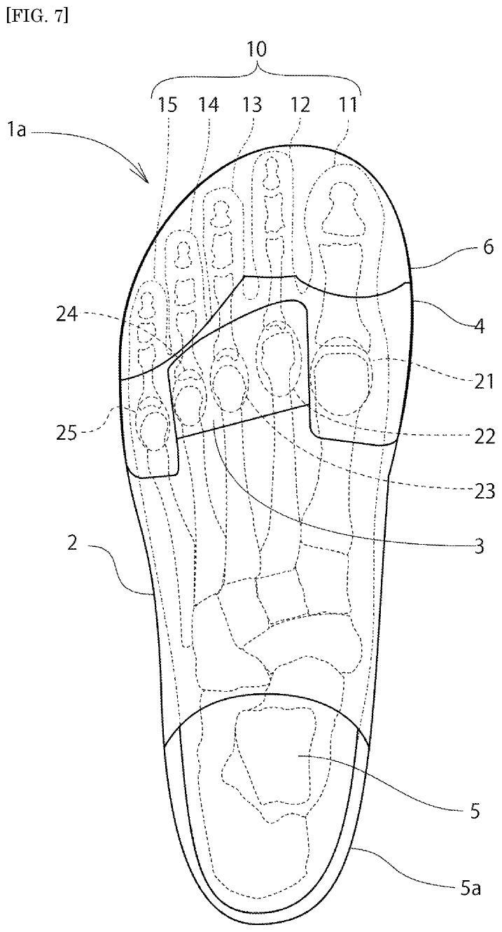

The toe supporting portion 6 of the shoe insole 1a according to the present embodiment is provided at the top end portion of the shoe insole 1a. As shown in FIGS. 5, 6A and 6B, the toe supporting portion 6 is provided on the top layer, the upper cushioning portion 4 is provided under the toe supporting portion 6, and the lower cushioning portion 3 is provided under the upper cushioning portion 4, thereby forming a level difference. Accordingly, as shown in FIG. 7, the toe 10 is supported by the toe supporting portion 6, the first toe ball 21 and the fifth toe ball 25 are supported by the upper cushioning portion 4, and the second toe ball 22, the third toe ball 23 and the fourth toe ball 24 are supported by the lower cushioning portion 3.

As described above, the shoe insole 1a of the present embodiment ensures effects same as those of the shoe insole 1 according to one embodiment of the present invention. Moreover, by providing the toe supporting portion 6 for supporting the toe 10 on the upper cushioning portion 4, it becomes easier to obtain the sense of kicking the ground with the toes 10 during walking or the sense of grasping the ground with the toes 10 in a standing posture, thereby improving stability of the posture and reducing fatigue in the sole.

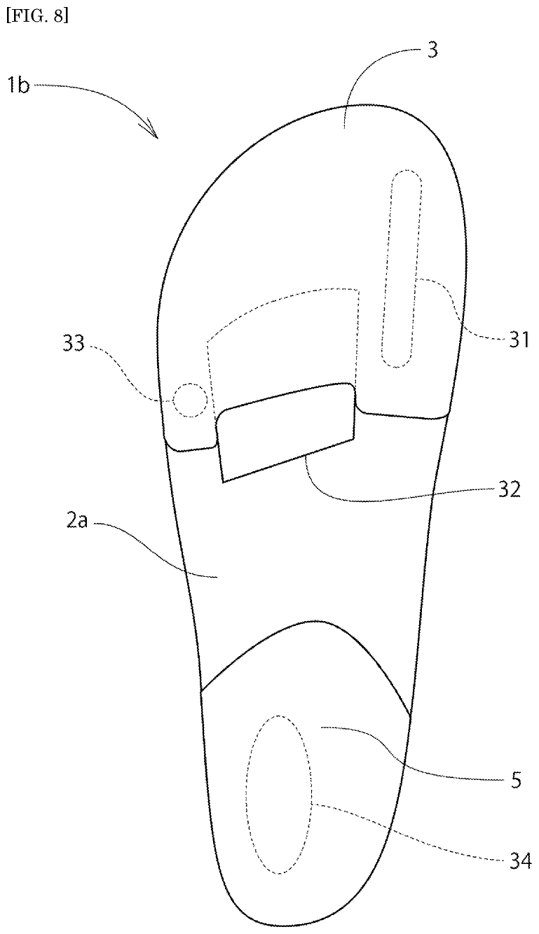

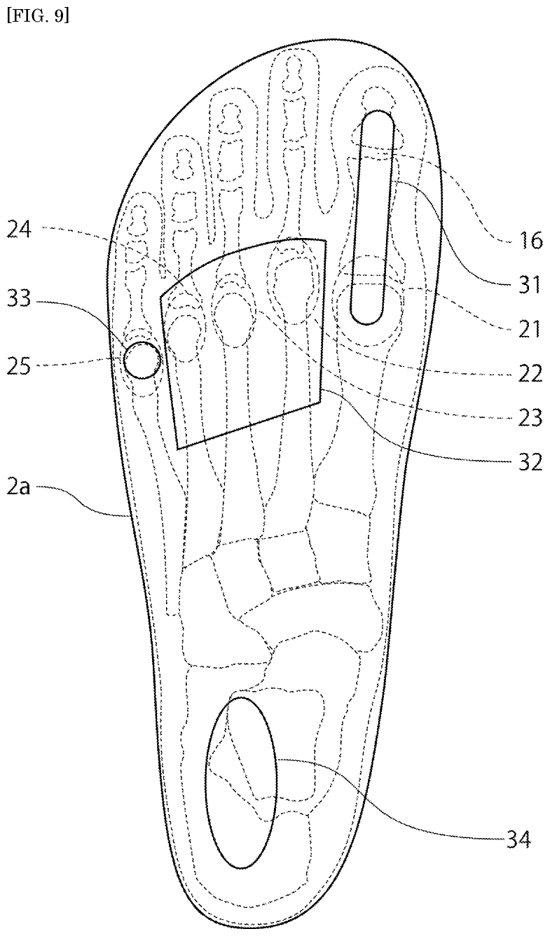

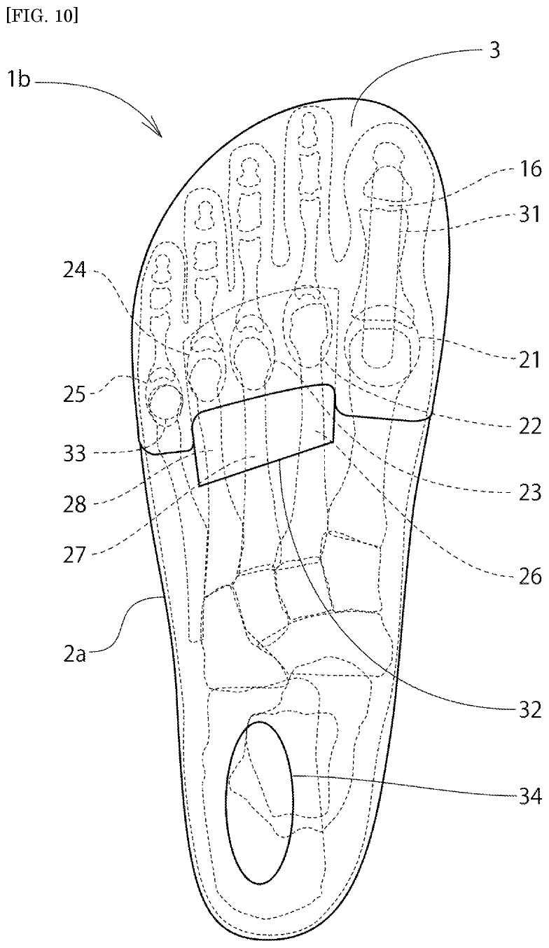

FIGS. 8 to 10 show a shoe insole having an insole main body in which through-holes are formed at predetermined portions (hereinafter referred to as “a perforated type shoe insole”) as another embodiment of the shoe insole of the present invention. The perforated type shoe insole 1b according to the present embodiment includes an insole main body 2a having a substantially foot shape, a lower cushioning portion 3 provided on the insole main body 2a in a front portion thereof for supporting a part from the tiptoe to the position of the metatarsal head part, and a heel supporting portion 5 provided on the insole main body 2a in a back portion thereof for supporting a part corresponding to the position of the heel. Furthermore, as shown in FIG. 9, the insole main body 2a of the shoe insole 1b of the present embodiment is characterized by including a first toe hole 31 formed in a portion from the IP joint 16 to the position of the first toe ball 21, a MP joint hole 32 formed in a portion corresponding to the positions of at least the second toe ball 22, the third toe ball 23 and the fourth toe ball 24, a fifth toe ball hole 33 formed in a portion corresponding to the position of the fifth toe ball 25, and a heel hole 34 formed in a portion corresponding to the position of the calcaneus center.

The lower cushioning portion 3, which is provided on the insole main body 2a in a front portion thereof in the shoe insole 1b according to the present embodiment, has a flat shape (see FIG. 3A), and supports a part from the tiptoe to the position of the metatarsal head part, namely the toe 10, the first toe ball 21, the second toe ball 22, the third toe ball 23, the fourth toe ball 24 and the fifth toe ball 25.

Further, the heel supporting portion 5 provided in a back portion of the insole main body 2a according to the present embodiment supports the part corresponding to the position of the heel. Similar to the lower cushioning portion 3, the heel supporting portion 5 may be formed in a flat shape; however, the heel supporting portion 5 may have a thick portion that wraps around the heel to provide a stable support for the heel.

By providing the lower cushioning portion 3 on the insole main body 2a according to the present embodiment in a front portion thereof, a portion corresponding to the toe 10, the first toe ball 21, the second toe ball 22, the third toe ball 23, the fourth toe ball 24 and the fifth toe ball 25 is supported, the first toe hole 31 is positioned at the position from the IP joint 16 to the first toe ball 21, the MP joint hole 32 is positioned at the position corresponding to the second toe ball 22, the third toe ball 23 and the fourth toe ball 24, and the fifth toe ball hole 33 is positioned at the position of the fifth toe ball 25 (see FIG. 9).

Further, by providing the heel supporting portion 5 on the insole main body 2a in a back portion of the insole main body 2a according to the present embodiment, a portion corresponding to the position of the heel is supported and the heel hole 34 is positioned at the position of the calcaneus center.

In contrast to the previously-known structure for directly supporting arches, the perforated type shoe insole 1b according to the present embodiment with the structure described above has the lower cushioning portion 3 and the heel supporting portion 5 both having a flat shape. Therefore, the shoe insole 1b ensures an effect of allowing the wearers to maintain a sense of balance by relying on the sensation in the sole of the foot, or to enhance the sense of balance and correct the standing and walking postures by themselves, thereby preventing fallen arches or restoring and correcting the arches.

Further, in the shoe insole 1b of the present embodiment, by providing the lower cushioning portion 3 on the insole main body 2a, the toe 10, the first toe ball 21, the second toe ball 22, the third toe ball 23, the fourth toe ball 24, and the fifth toe ball 25 are supported directly by the upper face of the lower cushioning portion 3, and the lower cushioning portion 3 absorbs shocks from the ground, thereby reducing shocks applied to the toe 10, the first toe ball 21, the fifth toe ball 25, and the like during walking.

Further, in this embodiment of the shoe insole 1b, by providing the first toe hole 31, the MP joint hole 32, the fifth toe ball hole 33 and the heel hole 34 at predetermined portions of the insole main body 2a, it is possible to make the second toe ball 22, the third toe ball 23 and the fourth toe ball 24, which protrude to the backside of the foot to a greater extent than the first toe ball 21 and the fifth toe ball 25 during walking or standing on tiptoe, sink into the MP joint hole 32. Accordingly, it becomes possible to support the toe 10, the first toe ball 21, the second toe ball 22, the third toe ball 23, the fourth toe ball 24, and the fifth toe ball 25 in a good balance, thereby preventing pain in the sole of the foot even during prolonged walking, thus suppressing gait disturbance and arthropathy.

In addition, during the standing posture, the first toe ball 21 sinks slightly into the first toe hole 31, the second toe ball 22, the third toe ball 23 and the fourth toe ball 24 sink slightly into the MP joint hole 32, the fifth toe ball 25 sinks slightly into the fifth toe ball hole 33, and the heel part sinks slightly into the heel hole 34. Accordingly, the anterior portion of the sole and the heel portion can be stably supported, thereby alleviating or preventing pain in the sole.

Further, during exercise, particularly upon the start or the end of exercise, a big load is applied to the first toe 11 and the first toe ball 21. However, in the shoe insole 1b of the present embodiment, by forming the first toe hole 31 in the insole main body 2a, the load applied to the first toe 11 and the first toe ball 21 is greatly reduced, thereby alleviating or preventing pain in the sole.

Furthermore, compared with the shoe insole 1 according to the embodiment of lamination type, the perforated type shoe insole 1b according to the present embodiment can be made thinner.

Also for the perforated type shoe insole 1b according to the present embodiment, the heights (thicknesses) of the lower cushioning portion 3 and the heel supporting portion 5 are not particularly limited. However, the height of the upper face of the lower cushioning portion 3 (i.e., the thickness of the lower cushioning portion 3) is preferably substantially the same as the height of the upper face of the heel supporting portion 5 (i.e., the thickness of the heel supporting portion 5). By thus setting the height of the upper face of the lower cushioning portion 3 to a height substantially identical to the height of the upper face of the heel supporting portion 5, it becomes possible to further enhance an effect of preventing fallen arches, maintaining a sense of balance, and correcting the postures.

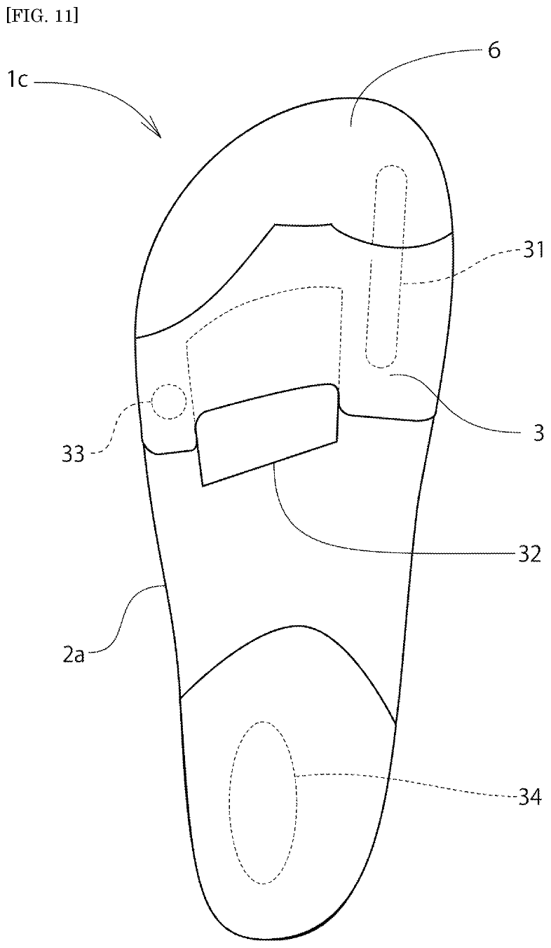

As another embodiment of the perforated type shoe insole of the present invention, the shoe insole 1c shown in FIG. 11 has a fundamental structure similar to that of the shoe insole 1b according to the embodiment described above, i.e., the shoe insole 1c is configured such that the first toe hole 31, the MP joint hole 32, the fifth toe ball hole 33, and the heel hole 34 are formed at predetermined portions of the insole main body 2a having a substantially foot shape, and the lower cushioning portion 3 is provided on the insole main body 2a in a front portion thereof, and the heel supporting portion 5 is provided on the insole main body 2a in a back portion thereof. Further, the shoe insole 1c of the present embodiment is characterized by including a toe supporting portion 6 for supporting the toe 10 on the lower cushioning portion 3.

The toe supporting portion 6 of the shoe insole 1c of the present embodiment is provided at the top end portion of the shoe insole 1c. The toe supporting portion 6 is provided on the lower cushioning portion 3, thereby generating a level difference. Accordingly, the toe 10 is planarly supported by the toe supporting portion 6, and the first toe ball 21, the second toe ball 22, the third toe ball 23, the fourth toe ball 24, and the fifth toe ball 25 are supported by the lower cushioning portion 3.

As described above, the shoe insole 1c of the present embodiment ensures effects same as those of the shoe insole 1b according to one embodiment of the present invention. Moreover, by providing the toe supporting portion 6 for supporting the toe 10 on the lower cushioning portion 3, it becomes easier to obtain the sense of kicking the ground with the toes 10 during walking or the sense of grasping the ground with the toes 10 in a standing posture, thereby improving stability of the posture and reducing fatigue in the sole.

The various embodiments of the shoe insole of the present invention have been described in detail above. However, all of the lower cushioning portion 3, the upper cushioning portion 4, the heel supporting portion 5 and the toe supporting portion 6 of the shoe insole 1 or 1a according to the embodiment of lamination type shoe insole described above, and the lower cushioning portion 3, the heel supporting portion 5 and the toe supporting portion 6 of the shoe insole 1b or 1c according to the embodiment of perforated type shoe insole described above may be configured to be detachable. By configuring the lower cushioning portion 3, the upper cushioning portion 4, the heel supporting portion 5, and the toe supporting portion 6 to be detachable, these portions can be easily adjusted by being individually replaced with one having a different thickness according to the shape and the condition of the foot or according to the user's preference.

By providing the lower cushioning portion 3, the upper cushioning portion 4, the heel supporting portion 5, and the toe supporting portion 6, each of which is detachable, according to the present embodiment in the existing flat shoe insoles as necessary, the same effects as those of the shoe insoles of the present invention, especially the lamination type shoe insoles, can be obtained.

With regard to the embodiment of lamination type shoe insole 1 of the present invention, the insole main body 2, the lower cushioning portion 3, the upper cushioning portion 4, and the heel supporting portion 5 may be integrally molded. For the shoe insole 1a, the insole main body 2, the lower cushioning portion 3, the upper cushioning portion 4, the heel supporting portion 5, and the toe supporting portion 6 may be integrally molded. Similarly, with regard to the embodiment of perforated type shoe insole 1b of the present invention, the insole main body 2b, the lower cushioning portion 3, and the heel supporting portion 5 may be integrally molded. For the shoe insole 1c, the insole main body 2b, the lower cushioning portion 3, the heel supporting portion 5, and the toe supporting portion 6 may be integrally molded.

With regard to a shoe with an insole structure of the present invention, an embodiment of the shoe with a lamination type insole structure has an insole structure in which the lower cushioning portion, the upper cushioning portion, and the heel supporting portion according to the shoe insole of the present invention are provided on the upper surface of the middle sole of the shoe, and an embodiment of a shoe with a perforated type insole structure has a structure in which the first toe hole, the MP joint hole, the fifth toe ball hole and the heel hole are formed on the middle sole of the shoe and the lower cushioning portion and the heel supporting portion are provided on the middle sole of the shoe having these holes. These structures are substantially the same as that of the shoe insoles of the present invention. The lower cushioning portion, the upper cushioning portion, and the heel supporting portion provided on the upper face of the middle sole may be detachable.

For the embodiment of the shoe with a lamination type shoe insole structure, the middle sole, the lower cushioning portion, the upper cushioning portion, and the heel supporting portion may be integrally molded. In other words, the lower cushioning portion and the upper cushioning portion may be integrally molded in a front portion of the upper face of the middle sole and the heel supporting portion may be integrally molded in a back portion of the upper face of the middle sole. Also for the embodiment of a shoe with a perforated type shoe insole structure, the lower cushioning portion and the heel supporting portion may be integrally molded on the middle sole of the shoe on which the first toe hole, the MP joint hole, the fifth toe ball hole, and the heel hole are formed.

The shoe with a lamination type shoe insole structure according to the present embodiment having the structure described above also has the lower cushioning portion and the upper cushioning portion having a flat shape in contrast to the previously-known structure for directly supporting arches. Therefore, it is possible to ensure an effect of allowing the wearers to maintain a sense of balance by relying on the sensation in the sole of the foot, or to enhance the sense of balance and correct the standing and walking postures by themselves, thereby preventing fallen arches or restoring and correcting the arches.

In addition, with regard to the embodiment of the shoe with a lamination type shoe insole structure of the present invention, it is preferable to set the height (thickness) of the lamination of the lower cushioning portion and the upper cushioning portion provided on the upper face of the middle sole to be substantially identical to the height of the upper face of the heel supporting portion (thickness of the heel supporting portion). On the other hand, with regard to the embodiment of the shoe with a perforated type shoe insole structure of the present invention, it is preferable to set the height (thickness) of the lower cushioning portion provided on the upper face of the middle sole on which the first toe hole and the like are formed at predetermined positions to be substantially identical to the height of the upper face of the heel supporting portion (thickness of the heel supporting portion). With these configurations, it becomes possible to further enhance an effect of preventing fallen arches, maintaining a sense of balance, and correcting the postures.

Furthermore, in the embodiment of the shoe with an insole structure of the present invention, the toe supporting portion may be provided to support the toe in both of the lamination type shoe insole and the perforated type shoe insole. The toe supporting portion may be integrally molded with the upper cushioning portion for the lamination type shoe insole, and may be integrally molded with the lower cushioning portion for the perforated type shoe insole. By providing the toe supporting portion in the embodiment of the shoe with an insole structure of the present invention, it becomes easier to obtain the sense of kicking the ground with the toes during walking or the sense of grasping the ground with the toes in a standing posture, thereby providing shoes capable of improving stability of the posture and reducing fatigue in the sole.

The embodiments of the shoe insole and the shoe with an insole structure of the present invention described above in detail must not be interpreted as substantially limiting the technical concept of the present invention. For example, the outer shapes of the lower cushioning portion 3, the upper cushioning portion 4, the heel supporting portion 5, and the toe supporting portion 6 illustrated in each embodiment is merely an example. In particular, the outer shapes of the tiptoe portions of the lower cushioning portion 3, the upper cushioning portion 4, and the toe supporting portion 6 are preferably adjusted according to the shape of the user's foot (Greek, Egyptian, Square, or the like).

The size (length) of the MP joint hole formed in the insole main body of the perforated type shoe insole of the present invention, or in the middle sole of the shoe with a perforated type shoe insole structure is not limited, insofar as the MP joint hole is formed at least in the portion corresponding to the positions of the second toe ball, the third toe ball and the fourth toe ball. However, to allow the sole to easily fit to the shape of the user's foot, the MP joint hole may be formed in a portion from the positions of the second toe ball 22, the third toe ball 23 and the fourth toe ball 24 to the portions corresponding to the middle of the second metatarsal 26, the third metatarsal 27, and the fourth metatarsal 28, as in the MP joint hole 32 shown in FIG. 9. The present invention can be implemented with appropriate improvement, modification, and/or addition by creativity and ingenuity of those skilled in the art without departing from the scope of the invention.

### REFERENCE NUMERALS

1,1a,1b,1c: Shoe Insole
2,2a: Insole Main Body
3: Lower Cushioning Portion
4: Upper Cushioning Portion
5: Heel Supporting Portion
5a: Thick Portion
5b: Upper Face of Heel Supporting Portion
6: Toe Supporting Portion
10: Toe
11: First Toe
12: Second Toe
13: Third Toe
14: Fourth Toe
15: Fifth Toe
16: IP Joint
21: First Toe Ball
22: Second Toe Ball
23: Third Toe Ball
24: Fourth Toe Ball
25: Fifth Toe Ball
26: Second Metatarsal
27: Third Metatarsal
28: Fourth Metatarsal
31: First Toe Hole
32: MP Joint Hole
33: Fifth Toe Ball Hole
34: Heel Hole

1. A shoe insole comprising:
an insole main body having a substantially foot shape;
a lower cushioning portion provided on the insole main body in a front portion for supporting a part from the tiptoe to the position of the metatarsal head part;
an upper cushioning portion having a substantially U shape provided on the lower cushioning portion for supporting a part corresponding to the positions of the toe, the first toe ball, and the fifth toe ball; and
a heel supporting portion provided on the insole main body in a back portion for supporting a part corresponding to the position of the heel.

2. The shoe insole according to claim 1, wherein the height of a lamination of the lower cushioning portion and the upper cushioning portion is substantially identical to the height of an upper face of the heel supporting portion.

3. The shoe insole according to claim 1, which comprises a toe supporting portion provided on the upper cushioning portion for supporting the toe.

4. A shoe insole comprising:
an insole main body having a substantially foot shape;
a lower cushioning portion provided on the insole main body in a front portion for supporting a part from the tiptoe to the position of the metatarsal head part;
a heel supporting portion provided on the insole main body in a back portion for supporting a part corresponding to the position of the heel;
the insole main body comprising:
a first toe hole formed in a part from the IP joint to the position of the first toe ball;
a MP joint hole formed in a part corresponding to at least the positions of the second toe ball, the third toe ball, and the fourth toe ball;
a fifth toe ball hole formed in a part corresponding to the position of the fifth toe ball; and
a heel hole formed in a part corresponding to the position of the calcaneus center.

5. The shoe insole according to claim 4, wherein the height of an upper face of the lower cushioning portion is substantially identical to the height of an upper face of the heel supporting portion.

6. The shoe insole according to claim 4, which comprises a toe supporting portion provided on the lower cushioning portion for supporting the toe.

7. A shoe comprising the shoe insole according to claim 1, comprising:
the lower cushioning portion provided in a front portion of an upper face of a middle sole for supporting the part from the tiptoe to the position of the metatarsal head part;
the upper cushioning portion having the substantially U shape provided on the lower cushioning portion for supporting the part corresponding to the positions of the toe, the first toe ball, and the fifth toe ball; and
the heel supporting portion provided in a back portion of the upper face of the middle sole for supporting the part corresponding to the position of the heel.

8. The shoe with an insole structure according to claim 7, wherein the height of a lamination of the lower cushioning portion and the upper cushioning portion is substantially identical to the height of an upper face of the heel supporting portion.

9. The shoe with an insole structure according to claim 7, which comprises a toe supporting portion provided on the upper cushioning portion for supporting a part corresponding to the position of the toe.

10. A shoe comprising the shoe insole according to claim 4, comprising:
the lower cushioning portion provided in a front portion of an upper face of a middle sole for supporting the part from the tiptoe to the position of the metatarsal head part;
the heel supporting portion provided in a back portion of the upper face of the middle sole for supporting the part corresponding to the position of the heel,
the middle sole comprising:
the first toe hole formed in a part from the IP joint to the position of the first toe ball;
the MP joint hole formed in the part corresponding to at least the positions of the second toe ball, the third toe ball, and the fourth toe ball;
the fifth toe ball hole formed in the part corresponding to the position of the fifth toe ball; and
the heel hole formed in the part corresponding to the position of the calcaneus center.

11. The shoe with an insole structure according to claim 10, wherein the height of an upper face of the lower cushioning portion is substantially identical to the height of an upper face of the heel supporting portion.

12. The shoe with an insole structure according to claim 10, which comprises a toe supporting portion provided on the lower cushioning portion for supporting a part corresponding to the position of the toe.
